(19) United States
(12) Patent Application Publication
(10) Pub. No.: US 2025/0166725 A1
(43) Pub. Date: May 22, 2025

(54) DISCOVERY SYSTEM FOR CANDIDATES BASED ON TARGET PROTEIN STRUCTURES AND ITS OPERATION METHOD

(71) Applicant: CALICI CO., LTD., Daejeon (KR)

(72) Inventors: Jae Mun CHOI, Daejeon (KR); Young Bin PARK, Seoul (KR); Chul SUNG, White Plains, NY (US); Van Huong LE, Daejeon (KR); Trong Tue TRAN, Daejeon (KR); Yu Kyung YUN, Daejeon (KR); Jin Hee PARK, Daejeon (KR); Jonathan WILLIANTO, White Plains, NY (US); Nuzup SHADIEV, Seoul (KR)

(73) Assignee: CALICI CO., LTD., Daejeon (KR)

(21) Appl. No.: 18/878,969

(22) PCT Filed: Jan. 5, 2024
(86) PCT No.: PCT/KR2024/000313

§ 371 Date: Dec. 26, 2024

(30) Foreign Application Priority Data
Jul. 26, 2023 (KR) 10-2023-0097539
Dec. 29, 2023 (KR) 10-2023-0197930

(51) Int. Cl. G16B 15/30; G16B 5/00

(52) U.S. Cl. CPC G16B 15/30; G16B 5/00

(57) ABSTRACT

A method for operating a protein structure-based candidate discovery system may include: displaying a first screen for receiving input of a plurality of ligands to be docked to a target protein; calculating predicted binding energy by sequentially performing molecular docking on the plurality of ligands based on settings input by a user via the first screen; displaying a first list on a second screen, the first list containing rows including a user interface control for user selection, names of the plurality of ligands, and the calculated binding energy values; predicting binding affinity for the rows selected by the user via the user interface control from the first list displayed on the second screen; and displaying a second list on a third screen, the second list containing rows including the names of the plurality of ligands, values of the binding energy, and values of the predicted binding affinity.

### TECHNICAL FIELD

Disclosure relates to a discovery system for candidates based on protein structures and its operation method, and more specifically, to a discovery system for candidates based on target protein structures for predicting binding affinity and its operation method.

### BACKGROUND ART

Proteins possess limited degrees of freedom and adopt specific three-dimensional structures determined by their constituent amino acid sequences. The function of a protein is determined by its unique three-dimensional structure. Therefore, once a target protein is identified, it is possible to search for candidates that bind to the active site of the specific protein and modulate its function by targeting its unique structure. Substances designed based on such structural approaches can shorten development time and enable the creation of substances that act exclusively on the target protein, thereby minimizing unwanted effects, such as side effects.

Such target protein structure-based candidate searches are commonly used for the discovery of drug candidates. This is because the nature of drug development requires reducing the time and cost associated with the initial lead compound or candidate discovery, while ensuring minimal side effects—an essential characteristic in this field. The discovery of drug candidates represents an early stage in drug development, and structure-based in silico screening methods are gaining attention. Protein structure-based in silico screening identifies potential drug candidates from compound databases based on the three-dimensional structure of proteins to which the drug candidates could bind. The term “in silico” refers to computer-based simulations or virtual experiments using computer programming. Through in silico screening, the interactions between proteins and compounds can be simulated to predict the binding strength of compounds. Protein structure-based in silico screening offers advantages over ligand-based screening, which is dependent on known ligands and limited in identifying entirely novel chemical structures. By targeting the active site of a protein, this approach not only identifies compounds that fit precisely but is also useful for exploring new chemical spaces to discover novel compounds.

In the discovery of candidates based on protein structures, where such principles are applied, there is a growing demand to integrate big data analysis technologies and artificial intelligence technologies with billions of compounds in chemical libraries, utilizing various analytical tools concurrently. To meet this demand, research laboratories in pharmaceutical companies or academic institutions have been installing and operating a variety of software. For example, the European Molecular Biology Laboratory (EMBL) develops and provides various tools for bioinformatics, which can be downloaded or accessed via simple web applications.

Even when candidates based on protein structures are identified, it has been challenging to immediately proceed to the development stages for commercialization. For instance, once a drug candidate is discovered, it undergoes various cell and animal experiments for validation and optimization, ultimately culminating in clinical trials for human application. However, even if a drug candidate is identified, users with biological knowledge but lacking familiarity with computational technologies face difficulties in directly applying the discovery results to clinical trials. This is primarily because the discovery results provided by drug candidate software typically do not include experimental concentration values. In such cases, additional experiments are required to determine the experimental concentration values after discovering the drug candidates. These additional experiments generally involve serial dilution of candidate drugs, performing numerous repeated experiments, and analyzing the results to determine the therapeutic concentration range deemed effective. As a result, this process is both time-consuming and costly.

### DISCLOSURE

### Technical Problem

A technical problem is to provide a protein structure-based candidate discovery system that, in addition to identifying candidates based on protein structures, predicts the binding affinity between target proteins and ligands and delivers these results to users through a cloud platform.

Another technical problem is to provide a drug candidate discovery system that, in addition to identifying drug candidates, predicts the binding affinity between target proteins and ligands and delivers these results to users through a cloud platform.

### Technical Solution

A method for operating a protein structure-based candidate discovery system according to an embodiment, implemented as a cloud platform to provide functionalities or services required for protein structure-based candidate discovery in the form of a web service, may include: displaying a first screen for receiving input of a plurality of ligands to be docked to a target protein; calculating predicted binding energy by sequentially performing molecular docking on the plurality of ligands based on settings input by a user via the first screen; displaying a first list on a second screen, the first list comprising rows including a user interface control for user selection, names of the plurality of ligands, and the calculated binding energy values; predicting binding affinity corresponding to protein-ligand binding poses used in the binding energy calculation for the rows selected by the user via the user interface control from the first list displayed on the second screen; and displaying a second list on a third screen, the second list comprising rows including the names of the plurality of ligands, values of the binding energy, and values of the predicted binding affinity.

In some embodiments, the values of the binding energy may be displayed in a unit representing the amount of energy, and the values of the binding affinity may be displayed in a unit of molar concentration.

In some embodiments, the values of the binding energy may be displayed in a unit of Kcal/mol, and the values of the binding affinity may be displayed in a unit of fM, pM, nM, μM, mM, or M.

In some embodiments, wherein the displaying the second list on the third screen may include: displaying the rows constituting the second list on the third screen, sorted in descending or ascending order based on the values of the binding affinity.

In some embodiments, the binding affinity may be predicted using an artificial intelligence model, the artificial intelligence model being trained with experimental measurement data including at least one of a dissociation constant (Kd), an inhibition constant (Ki), and a half maximal inhibitory concentration (IC50).

In some embodiments, the artificial intelligence model may be trained using the experimental measurement data and binding structure data of the target protein and the ligand as training data.

In some embodiments, the artificial intelligence model may include a convolution layer part including filters for encoding patterns to be identified for predicting the binding affinity between the target protein and the ligand, and a dense layer part for integrating features extracted through the convolution layer part.

In some embodiments, the convolution layer part may include three convolution layers, each having 64, 128, and 256 filters, respectively.

In some embodiments, the dense layer part may include three dense layers, each having 1000, 500, and 200 neurons, respectively.

In some embodiments, the artificial intelligence model may include a CNN(Convolutional Neural Network) model or a ResNet 3D (Residual Network 3D) model.

A protein structure-based candidate discovery system according to an embodiment, implemented as a cloud platform to provide functionalities or services required for protein structure-based candidate discovery in the form of a web service, may include: a project management module configured to create a project for adding a task to perform the discovery of a protein structure-based candidate; a simulation management module configured to create a simulation desired by a user on the created project; a simulation setting module configured to set a simulation workflow for the simulation based on user input using a simulation setting area, the simulation setting area comprising a task module selection area, the task module selection area comprising: a first object, which is dragged and dropped onto a canvas area and converted into a first node that calculates predicted binding energy based on docking between a target protein and a ligand, and a second object, which is dragged and dropped onto the canvas area and converted into a second node that predicts binding affinity corresponding to the protein-ligand binding pose used in the binding energy calculation performed by the first node; and a simulation workflow management module configured to manage information on nodes that can precede or follow in the simulation workflow.

In some embodiments, when a run button displayed on the first node is clicked, a first screen for receiving input of a plurality of ligands to be docked to the target protein may be displayed.

In some embodiments, the first node may be configured to calculate predicted binding energy by sequentially performing molecular docking on the plurality of ligands based on settings input by the user via the first screen.

In some embodiments, after the calculation of the binding energy is completed, a first list comprising rows including a user interface control for user selection, names of the plurality of ligands, and the calculated binding energy values may be displayed on a second screen.

In some embodiments, the second node may be configured to predict binding affinity corresponding to protein-ligand binding poses used in the binding energy calculation for the rows selected by the user via the user interface control from the first list displayed on the second screen.

In some embodiments, after the prediction of the binding affinity is completed, a second list comprising rows including the names of the plurality of ligands, values of the binding energy, and values of the binding affinity may be displayed on a third screen.

In some embodiments, the second list may be displayed on the third screen by displaying the rows constituting the second list on the third screen, sorted in descending or ascending order based on the values of the binding affinity.

In some embodiments, the values of the binding energy may be displayed in a unit of Kcal/mol, and the values of the binding affinity may be displayed in a unit of fM, pM, nM, μM, mM, or M.

In some embodiments, the binding affinity may be predicted using an artificial intelligence model, the artificial intelligence model being trained with experimental measurement data including at least one of a dissociation constant (Kd), an inhibition constant (Ki), and a half maximal inhibitory concentration (IC50).

In some embodiments, the simulation workflow management module may manage information on nodes that can precede or follow in the simulation workflow through metadata.

### Advantageous Effects

According to the embodiments, the system provides optimized functionalities and user interfaces for protein structure-based candidate discovery. It overcomes the limitations of conventional methods, where detailed tasks related to protein structure-based candidate discovery were performed using separate tools with low compatibility, making data sharing and consistent data management difficult. The system allows users to easily manage simulation workflows by creating, modifying, and deleting nodes.

In conjunction with this, the system predicts the binding affinity between target proteins and ligands and delivers this information to users, facilitating intuitive understanding of the results. Users can easily calculate the molecular weight of the identified candidates and the medium volume required for experiments, enabling direct application to cell experiments or clinical trials without the need for additional experiments. Consequently, the time and cost required to determine experimental concentrations for the candidates are significantly reduced. Furthermore, as these experimental concentrations are provided through a cloud platform, users can easily access the values from any location with internet connectivity. This also enhances collaboration among users, offering a convenient advantage.

### DESCRIPTION OF THE DRAWINGS

FIG. 1 is a block diagram illustrating a protein structure-based candidate discovery system according to an embodiment.

FIGS. 2 to 6 are diagrams showing example screens of a protein structure-based candidate discovery system according to an embodiment.

FIG. 7 is a diagram showing an example of simulation settings in a protein structure-based candidate discovery system according to an embodiment.

FIGS. 8 to 10 are diagrams illustrating an operation of a protein structure-based candidate discovery system according to an embodiment.

FIG. 11 is a diagram for explaining an operation method of a protein structure-based candidate discovery system according to an embodiment.

FIG. 12 is a diagram showing an implementation example of an artificial intelligence model of a protein structure-based candidate discovery system according to an embodiment.

FIG. 13 is a block diagram for explaining a computing device according to an embodiment.

### MODE FOR INVENTION

Hereinafter, the embodiments of the present invention will be described in detail with reference to the accompanying drawings so that those skilled in the art to which the present invention pertains can easily implement them. However, the present invention is not limited to the embodiments described herein and may be implemented in various different forms. Moreover, in order to clearly describe the present invention in the drawings, parts irrelevant to the description have been omitted, and similar reference numerals have been used for similar parts throughout the specification.

In the entire specification and claims, when a part is described as “including” a certain component, it means that, unless specifically stated otherwise, the inclusion of other components is not excluded and that other components may be further included.

Furthermore, the terms such as “ . . . part,” “ . . . unit,” and “ . . . module” described in the specification may refer to units capable of processing at least one function or operation as described herein, and these units may be implemented as hardware, software, or a combination of hardware and software.

In this specification, the terms “candidate” or “protein structure-based candidate” broadly encompass any substance derived using the target protein structure-based candidate discovery system provided herein, without being limited to a specific field. Preferably, it includes fields such as pharmaceutical development, food development, and livestock material discovery. More preferably, it refers to candidates in the pharmaceutical development field, but it is not limited thereto.

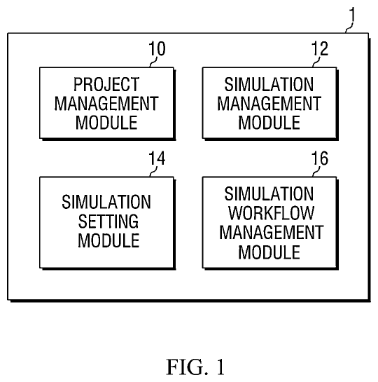

FIG. 1 is a block diagram illustrating a protein structure-based candidate discovery system according to an embodiment.

A protein structure-based candidate discovery system 1 according to an embodiment may be implemented as a cloud platform that provides functionalities or services required for protein structure-based candidate discovery to users in the form of a web service. Specifically, the protein structure-based candidate discovery system 1 may provide various functionalities or services, such as enabling a biologist with specific ideas for drug development to utilize in silico screening methods without requiring knowledge in other fields; detecting and removing errors (or defects) in protein structure files; efficiently detecting enzymatically active pockets for docking calculation (EAPDC) from protein structures and providing them to the user; providing real-time ranking of candidates based on docking binding energy while performing docking simulations, which require a significant amount of time; predicting docking binding energy in two stages to enhance reliability; and even validating the discovered candidates through collaboration with verification agencies.

The protein structure-based candidate discovery system 1 may provide the same functionalities or services to users in various environments through a web interface. Specifically, for example, some users may use mobile devices such as smartphones or tablet computers running a mobile operating system to receive services from the protein structure-based candidate discovery system 1, while other users may use a laptop computer running a Windows operating system to access the services. Additionally, other users may use a desktop computer running a Linux operating system to receive services from the protein structure-based candidate discovery system 1. In other words, the protein structure-based candidate discovery system 1, implemented as a cloud platform in the form of a web service, enables users in different environments to perform in silico candidate calculations using an artificial neural network and to utilize the same functionalities or services for subsequent experiments on the candidates, such as preclinical experiments. By doing so, the system enhances compatibility and user convenience and addresses several issues that previously required improvement in in silico calculations performed via terminal on Linux systems.

Referring to FIG. 1, the protein structure-based candidate discovery system 1 according to an embodiment may include a project management module 10, a simulation management module 12, a simulation setting module 14, and a simulation workflow management module 16.

The project management module 10 may create a project to which a series of tasks for performing the discovery of protein structure-based candidates may be added. Additionally, the project management module 10 may display the created project to the user through the display device of the computing device on which the protein structure-based candidate discovery system 1 is operating.

In some embodiments, the project management module 10 may perform project name encryption. For a project with project name encryption enabled, the project management module 10 may display the original, unencrypted project name to the user who created the project and to users authorized to access the project, while displaying the encrypted project name to users who do not have access rights to the project. Since project names may contain keywords related to protein structure-based candidate discovery that need to remain secure, preventing the project name from being directly exposed to users not involved in the same project on the cloud platform enhances security in the protein structure-based candidate discovery system 1, which is used by multiple users. Enabling or disabling project name encryption can be performed not only when the project is created but also by changing the option settings after the project has been created.

In some embodiments, the project management module 10 may support project participation through invitation codes. For example, when a user creates a project, the user may send an invitation code to other users, and the recipients of the invitation code may join the project by entering the code. In other words, users may participate in projects created by others through invitation codes. This enables users with knowledge in different fields to collaborate on a single project on the cloud platform to perform protein structure-based candidate discovery. Additionally, the project management module 10 may support permission settings for project members. For instance, the project management module 10 may grant administrator privileges to specific members among the project members. Furthermore, the project management module 10 may also allow a user participating in a project to leave the project if desired.

The simulation management module 12 may allow a user to create desired simulations on a project created by the project management module 10. A single simulation may include a plurality of task modules with specific functionalities for performing protein structure-based candidate discovery, and a single project may include multiple simulations. After creating a simulation, the user may upload protein structure data required to perform the plurality of task modules.

The simulation setting module 14 may display a simulation setting area 140 to the user through the display device of the computing device on which the protein structure-based candidate discovery system 1 is operating when the user selects one or more simulations managed by the simulation management module 12. In the simulation setting area 140, the user may configure the simulation workflow by arranging and connecting a plurality of task modules based on graph computing to perform the desired simulation. Specifically, the simulation setting area 140 may be laid out to allow the user to intuitively recognize functionalities for uploading protein structure data and task modules that perform detailed tasks related to candidate discovery based on the uploaded protein structure data. This configuration addresses the conventional issue where detailed tasks for protein structure-based candidate discovery were provided as separate tools with low compatibility, making data sharing and consistent data management challenging. The simulation setting area 140 may include a protein structure data input area 142, a task module selection area 144, and a canvas area 146.

The protein structure data input area 142 may include one or more objects that can be dragged and dropped onto the canvas area 146. For example, the protein structure data input area 142 may include a first object through a fourth object. The first object may be dragged and dropped onto the canvas area 146 and converted into a first node, which may receive protein structure data in the form of a PDB (Protein Data Bank) file from the user. The second object may be dragged and dropped onto the canvas area 146 and converted into a second node, which may receive protein structure data in the form of a PDB code from the user. The third object may be dragged and dropped onto the canvas area 146 and converted into a third node, which may receive protein structure data in the form of a protein sequence file from the user. The fourth object may be dragged and dropped onto the canvas area 146 and converted into a fourth node, which may receive protein structure data in the form of a protein sequence from the user.

The task module selection area 144 may also include one or more objects that can be dragged and dropped onto the canvas area 146. For example, the task module selection area 144 may include a fifth object through an eighth object. The fifth object may be dragged and dropped onto the canvas area 146 and converted into a fifth node, which may perform a task to identify optimal docking sites on the target protein structure. Specifically, the fifth node may utilize an artificial intelligence language model based on NLP (Natural Language Processing) to automatically identify active sites on the target protein and generate an optimal docking grid box. Additionally, the fifth node may automatically correct various errors that may exist in protein structure files (i.e., PDB files). In particular, the fifth node may detect and remove anisotropic B-factors in PDB files, detect alternative conformations in residue fields and modify them into non-alternative conformations, and detect unusual amino acids in residue fields to automatically modify them into one of the 20 standard amino acids. The sixth object may be dragged and dropped onto the canvas area 146 and converted into a sixth node, which may predict the tertiary structure of a protein from its amino acid sequence. The seventh object may be dragged and dropped onto the canvas area 146 and converted into a seventh node, which may analyze the binding energy (e.g., in kcal/mol) between a target protein and ligands and provide the results to the user in order of the most favorable binding. Specifically, the seventh node may perform grid-based in silico docking based on the input protein structure, determine chemical poses using the Lamarckian Genetic Algorithm (LGA), and calculate binding energy using an empirical scoring function. The eighth object may be dragged and dropped onto the canvas area 146 and converted into an eighth node, which may convert the predicted binding energy between the target protein and the ligand into binding affinity and perform comparative analysis. For example, the eighth node may use an artificial intelligence model trained on protein-ligand structures and Kd/Ki/IC50 values to predict binding affinity in units of molar concentration, such as fM, pM, nM, μM, mM, or M. The predicted concentration values may be displayed directly next to the binding affinity values to enable a person skilled in the art to intuitively recognize the necessary values.

The first through eighth objects can be dragged and dropped onto the canvas area 146 and converted into the first through eighth nodes, respectively. Users may freely arrange the first through eighth nodes on the canvas area 146 in any desired execution order according to the purpose and environment of the simulation. Additionally, users may establish connections between the first through eighth nodes placed on the canvas area 146 by setting edges. By arranging nodes and connecting edges between them, users can create candidate discovery simulations. Furthermore, as the number of nodes placed on the canvas area 146 increases and the edges between the nodes grow, resulting in increased complexity in the simulation workflow, users can manage protein structure data input information clearly and efficiently by creating, modifying, and deleting nodes.

In some embodiments, the simulation setting area 140 may further include an external module provision area 148. The external module provision area 148 may include a ninth object that can be dragged and dropped onto the canvas area 146 and converted into a ninth node with arbitrary functionalities provided from outside the protein structure-based candidate discovery system 1. This allows functionalities provided by other systems operating in conjunction with the protein structure-based candidate discovery system 1 to be easily incorporated into simulations by setting nodes in the canvas area 146.

As described above, users can click and drag desired objects from the protein structure data input area 142, the task module selection area 144, and the external module provision area 148, and drop them onto the canvas area 146 to place and freely move nodes. In some embodiments, node connection shapes may be displayed on the nodes placed in the canvas area 146. While the user clicks on a node connection shape displayed on the right side of a node, the color or shape of the node connection shapes displayed on the left side of other connectable nodes may change. The user cannot establish connections with nodes where the color of the node connection shapes on the left side remains unchanged and can only establish connections with nodes where the color of the node connection shapes on the left side has changed. This prevents users from creating invalid simulation workflows by allowing them to rely on the color changes of the node connection shapes to determine valid connections, without needing to be aware of whether causal relationships between the nodes can be established.

The information for determining whether connections between nodes are possible may be managed by the simulation workflow management module 16. The simulation workflow management module 16 may manage information about nodes that can precede or follow in the simulation settings and, if necessary, may utilize separate data structures, such as metadata. Additionally, the simulation workflow management module 16 may update the information, such as reflecting changes in the metadata, when the information about nodes that can precede or follow is modified.

While clicking on a node connection shape displayed on the right side of a node, a user may establish a connection to another node where the color of the node connection shape displayed on its left side has changed. The user may establish the connection either by clicking the node connection shape on one node and then clicking the node connection shape on another node or by clicking and dragging the node connection shape from one node to the node connection shape of another node. Once the connection is completed, a connection line is displayed between the nodes. The user may remove the connection by clicking the “X” displayed on the connection line.

Objects included in the task module selection area 144 may generate a run button within the node when converted into nodes on the canvas area 146. Users may click the run button to execute the task associated with the node. Before the task begins, the number of tokens required to perform the task may be displayed, and the task may proceed after the user confirms and the tokens are deducted. Once the task is completed, the run button may change to a result button, and a download button may additionally be generated. Users may click the result button to view the task results and click the download button to download the task results.

In this way, users may create a simulation workflow tailored for optimal compound development by configuring the relationships between nodes, each having specific functionalities, in the candidate discovery process. Additionally, even when new functionalities are added internally to the protein structure-based candidate discovery system 1 or introduced externally, nodes corresponding to the added functionalities may be generated, allowing users to easily establish connections with existing nodes.

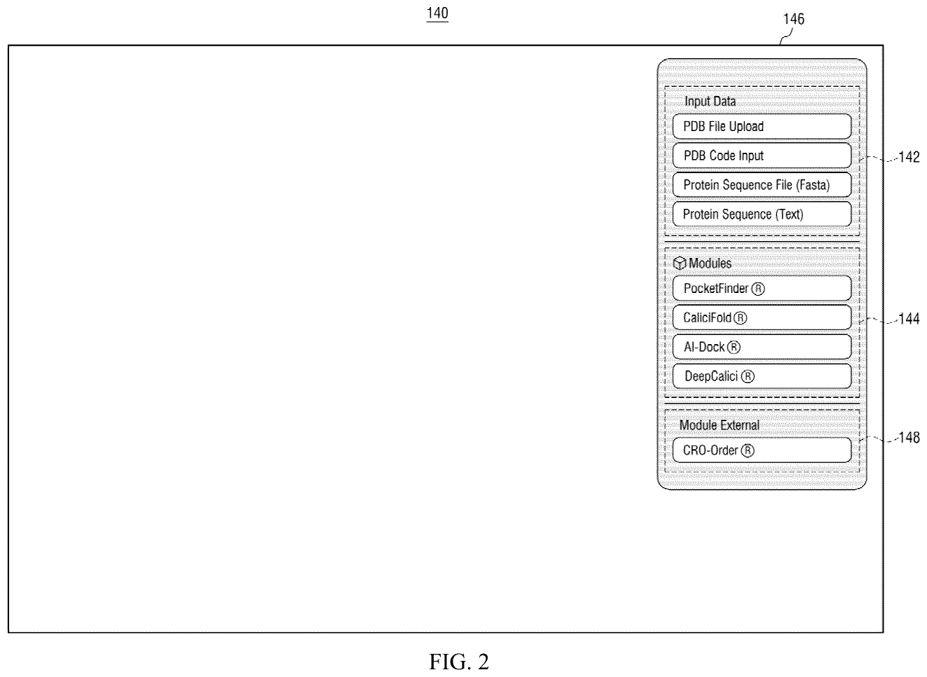

FIGS. 2 to 6 are diagrams showing example screens of a protein structure-based candidate discovery system according to an embodiment.

Referring to FIG. 2, the simulation setting area 140 may include a protein structure data input area 142, a task module selection area 144, a canvas area 146, and an external module provision area 148. As illustrated, the protein structure data input area 142 may include one or more objects related to functionalities for uploading protein structure data, the task module selection area 144 may include one or more objects related to detailed tasks for candidate discovery performed based on the uploaded protein structure data, and the external module provision area 148 may include one or more objects related to arbitrary functionalities provided externally. These objects may be dragged and dropped onto the canvas area 146 by the user and converted into nodes. The nodes may be connected by edges to form a graph, which represents the simulation workflow. Of course, the number or types of objects included in the task module selection area 144, the canvas area 146, and the external module provision area 148, as depicted in the drawings, are examples provided for illustrative purposes to explain the embodiments and are not intended to limit the scope of the invention to what is shown.

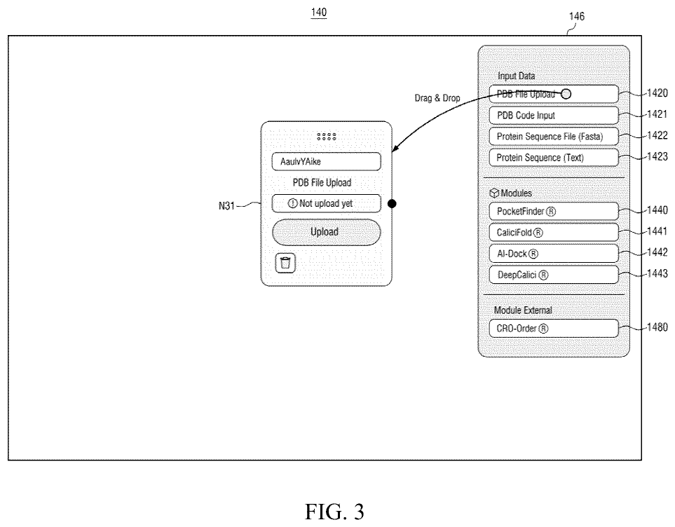

Referring to FIG. 3, the protein structure data input area 142 may include a first object 1420, a second object 1421, a third object 1422, and a fourth object 1423. The first object 1420, labeled “PDB File Upload,” is related to the functionality for receiving protein structure data in the form of PDB files. The second object 1421, labeled “PDB Code Input,” is related to the functionality for receiving protein structure data in the form of PDB codes. The third object 1422, labeled “Protein Sequence File (Fasta),” is related to the functionality for receiving protein structure data in the form of protein sequence files. The fourth object 1423, labeled “Protein Sequence (File),” is related to the functionality for receiving protein structure data in the form of protein sequences.

Meanwhile, the task module selection area 144 may include a fifth object 1440, a sixth object 1441, a seventh object 1442, and an eighth object 1443. The fifth object 1440, labeled “PocketFinder,” is related to the functionality for automatically identifying optimal docking sites. The sixth object 1441, labeled “CaliciFold,” is related to the functionality for predicting the tertiary structure of a protein from its amino acid sequence. The seventh object 1442, labeled “Al-Dock,” is related to the functionality for analyzing the binding energy between a target protein and ligands and automatically sorting them in the most favorable order. The eighth object 1443, labeled “DeepCalici,” is related to the functionality for converting the predicted binding energy between a target protein and a ligand into binding affinity and performing comparative analysis.

In some embodiments, the fifth object 1440 may automatically process PDB files containing protein structure data input by the user by detecting and removing anisotropic B-factors, detecting alternative conformations in residue fields and modifying them into non-alternative conformations, and detecting unusual amino acids in residue fields and modifying them into one of the 20 standard amino acids. For example, if docking simulations are performed without removing anisotropic B-factors from a PDB file, errors may occur, such as the inability to recognize the PDB file format or to read the PDB file. Similarly, if docking simulations are performed with alternative conformations or unusual amino acids present in the residue fields, errors may occur due to the presence of unknown amino acids. These issues may reduce the accuracy of in silico screening methods or increase the failure rate of candidate discovery. By automatically handling such error-inducing factors, the fifth object 1440 prevents inefficiencies and inaccuracies that may arise from users manually editing PDB files. It eliminates the need for collaboration with structural biologists and automates the preprocessing of PDB files internally, such that users do not need to be aware of the preprocessing steps. This allows users to focus entirely on candidate discovery, providing an efficient and streamlined environment for their work. Additionally, in some embodiments, the fifth object 1440 may perform modifications for missing residues in the protein structure of a PDB file. Specifically, it may inspect gaps between residues in the protein structure of the PDB file to detect missing residues. When missing residues are found, the fifth object 1440 may retrieve appropriate protein amino acid sequences from a sequence database to complete the missing residues and automatically fill in the missing residues using the retrieved protein amino acid sequences. As a result, subsequent tasks may be performed based on an error-free protein structure file, where potential errors in the simulation have been eliminated.

The fifth object 1440 may detect an enzymatically active pocket for docking calculation (EAPDC) from a protein structure file to determine the docking site. Specifically, the fifth object 1440 may predict docking sites (i.e., EAPDC) on the target protein structure using an artificial intelligence language model. Specifically, the fifth object 1440 may calculate the depth values of pockets based on the solvent-accessible surface (SAS) of the target protein's surface, generate a gradient class activation map for amino acids contributing to the prediction of the target protein's activity, and determine the docking site as the region of the target protein with the highest influence on its activity. This determination is made by considering the pocket depth values and the values of highly contributing amino acids identified in the gradient class activation map. Here, the gradient class activation map may be extracted from a graph convolutional network (GCN) trained using an enzyme commission (EC) number or gene ontology (GO) number, implemented in an embedding layer of a natural language processing model. The natural language processing model implemented in the embedding layer may be a transformer-based model.

Meanwhile, the external module provision area 148 includes a ninth object 1480. The ninth object 1480, labeled “CRO-Order,” is related to the functionality of sending a verification request for the desired candidate to a verification agency server.

As shown in FIG. 3, for example, the user may drag and drop the first object 1420, labeled “PDB File Upload,” onto the canvas area 146, where it may be converted into a node N31. The node N31 may include a button for receiving protein structure data in the form of PDB files. Additionally, the node N31 may display related information such as the identifier of the node and the task execution status. The node N31 may also include a button for deleting itself. As illustrated by the example of the node N31, the second object 1421 through the ninth object 1480 may also be dragged and dropped onto the canvas area 146, where they may be converted into nodes that display their unique buttons, information, and other features.

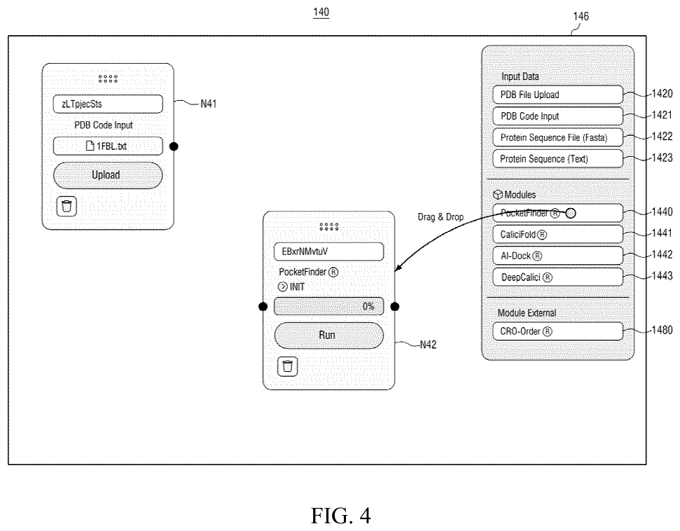

As shown in FIG. 4, the user may drag and drop the second object 1421, labeled “PDB Code Input,” onto the canvas area 146, where it may be converted into a node N41. The node N41 may include a button for receiving protein structure data in the form of PDB codes. Subsequently, the user may drag and drop the fifth object 1440, labeled “PocketFinder,” onto the canvas area 146, where it may be converted into a node N42. The node N42 may include a button for performing the functionality of automatically identifying optimal docking sites.

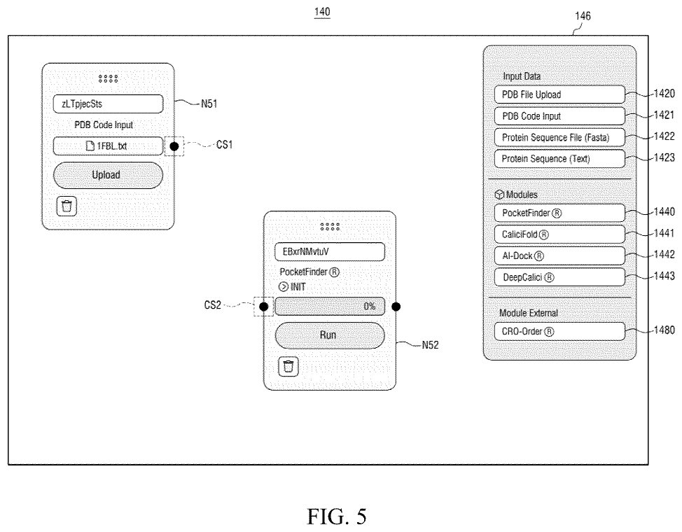

As shown in FIG. 5, node connection shapes may be displayed on nodes placed in the canvas area 146. Specifically, a node connection shape CS1 may be displayed on the right side of node N51, and a node connection shape CS2 may be displayed on the left side of node N52. While the user clicks on the node connection shape CS1 displayed on the right side of node N51, the color or shape of the node connection shape displayed on the left side of another node N52, which is connectable, may change. The user cannot establish connections with nodes where the color of the node connection shape on the left side has not changed and may only establish connections with nodes where the color of the node connection shape on the left side has changed. The change in the color or shape of the node connection shape CS1 displayed on the left side of a connectable node N52 while the user is clicking on the node connection shape CS1 of node N51 is determined based on information about nodes that can precede or follow, provided by the simulation workflow management module 16.

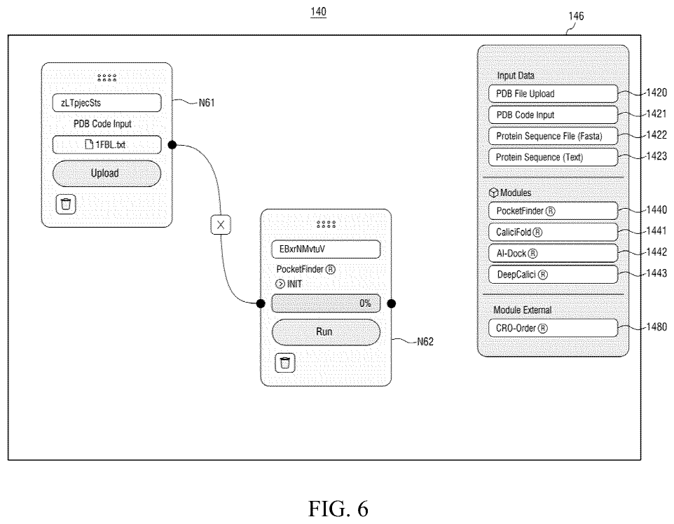

As shown in FIG. 6, while clicking on the node connection shape CS1 displayed on the right side of a node N61, the user may establish a connection to another node N62 where the color of the node connection shape CS2 displayed on its left side has changed. The connection can be established either by clicking the node connection shape CS1 of node N61 and then clicking the node connection shape CS2 of node N62, or by clicking and dragging the node connection shape CS1 of node N61 to the node connection shape CS2 of node N62. Once the connection is completed, a connection line is displayed between the nodes. The user can remove the connection by clicking the “X” displayed on the connection line.

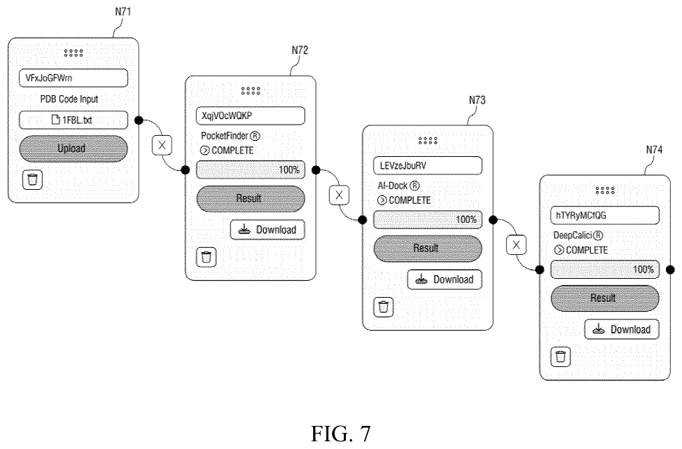

FIG. 7 is a diagram showing an example of simulation settings in a protein structure-based candidate discovery system according to an embodiment.

Referring to FIG. 7, an example of a created simulation workflow is shown with nodes N71 through N74. The node connection shape on the right side of node N71, which receives protein structure data in the form of PDB codes, is connected by an edge to the node connection shape on the left side of node N72, which automatically identifies optimal docking sites. Similarly, the node connection shape on the right side of node N72 is connected by an edge to the node connection shape on the left side of node N73, which analyzes and automatically sorts the binding energy between the target protein and ligands. Additionally, the node connection shape on the right side of node N73 is connected by an edge to the node connection shape on the left side of node N74, which converts the predicted binding energy between the target protein and ligands into binding affinity and performs comparative analysis. As explained earlier, users cannot establish connections to nodes where the color of the node connection shape on the left side has not changed. Connections can only be established to nodes where the color of the node connection shape on the left side has changed. This eliminates the need for users to consider whether nodes can precede or follow one another, thereby improving convenience in candidate discovery workflows.

In some embodiments, information about nodes that can precede or follow may be predetermined as follows.

TABLE 1

| Preceding Node | Node | Following Node |
|---|---|---|
| PDB File Upload | PocketFinder | Al-Dock |
| PDB Code Input | | |
| Protein Sequence File | | |
| (Fasta) | | |
| Protein Sequence (Text) | | |
| CaliciFold | | |
| Protein Sequence File | CaliciFold | PocketFinder |
| (Fasta) | | |
| Protein Sequence (Text) | | |
| PocketFinder | Al-Dock | DeepCalici |
| Al-Dock | DeepCalici | — |

For the node that automatically identifies optimal docking sites (“PocketFinder”), preceding nodes may include the node that receives protein structure data in the form of PDB files (“PDB File Upload”), the node that receives protein structure data in the form of PDB codes (“PDB Code Input”), the node that receives protein structure data in the form of protein sequence files (“Protein Sequence File (Fasta)”), the node that receives protein structure data in the form of protein sequences (“Protein Sequence (Text)”), and the node that predicts the tertiary structure of a protein from its amino acid sequence (“CaliciFold”). Following nodes may include the node that analyzes and automatically sorts the binding energy between the target protein and ligands (“Al-Dock”).

For the node that predicts the tertiary structure of a protein from its amino acid sequence (“CaliciFold”), preceding nodes may include the node that receives protein structure data in the form of protein sequence files (“Protein Sequence File (Fasta)”) and the node that receives protein structure data in the form of protein sequences (“Protein Sequence (Text)”), Following nodes may include the node that automatically identifies optimal docking sites (“PocketFinder”).

For the node that analyzes and automatically sorts the binding energy between the target protein and ligands (“Al-Dock”), preceding nodes may include the node that automatically identifies optimal docking sites (“PocketFinder”). Following nodes may include the node that converts the predicted binding energy between the target protein and ligands into binding affinity and performs comparative analysis (“DeepCalici”).

For the node that converts the predicted binding energy between the target protein and ligands into binding affinity and performs comparative analysis (“DeepCalici”), preceding nodes may include the node that analyzes and automatically sorts the binding energy between the target protein and ligands (“Al-Dock”).

Such information about nodes that can precede or follow may be managed by the simulation workflow management module 16, which may utilize separate data structures, such as metadata, if necessary. Additionally, the simulation workflow management module 16 may update the information, including reflecting changes in metadata when the information about nodes that can precede or follow is modified. In this way, a simulation workflow suitable for optimal compound development may be created by configuring the connections between nodes, each having specific functionalities, in the candidate discovery process.

In conventional simulation methods, conducting complex simulations for protein structure-based candidate discovery, which requires multiple attempts in various ways, demanded significant effort, time, and cost, while achieving satisfactory simulation settings was challenging. The graph computing-based simulation configuration method described through the embodiments improves upon conventional methods, enabling the intuitive and effortless creation and management of complex simulation workflows, as illustrated, while providing flexibility and convenience for easy modifications. It also allows the execution of highly intricate and complex simulation workflows. Additionally, it provides functionalities and user interfaces optimized for candidate discovery, addressing the challenges of the conventional approach, where detailed tasks related to candidate discovery were provided as separate tools with low compatibility, making data sharing and consistent data management difficult. By enabling the creation, modification, and deletion of nodes, the system allows for easy management of simulation workflows.

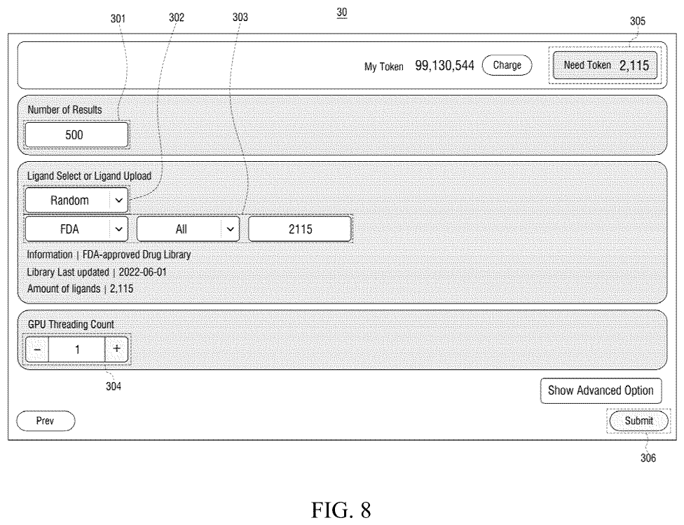

FIGS. 8 to 10 are diagrams illustrating an operation of a protein structure-based candidate discovery system according to an embodiment.

Referring to FIG. 8, a protein structure-based candidate discovery system according to an embodiment may display a first screen 30 for receiving input of a plurality of ligands to be docked to a target protein.

In this embodiment, the simulation setting module 14 of the protein structure-based candidate discovery system may display a simulation setting area 140 to the user. As previously described, the simulation setting area 140 may include a task module selection area 144 and a canvas area 146. The simulation setting module 14 may configure a simulation workflow based on the simulation set up by the user dragging and dropping multiple task modules onto the canvas area 146 and arranging and connecting them using a graph computing approach. Specifically, in this embodiment, the task module selection area 144 may include a first object and a second object. The first object may be dragged and dropped onto the canvas area 146 and converted into a first node that calculates predicted binding energy based on docking between the target protein and ligands. The second object may be dragged and dropped onto the canvas area 146 and converted into a second node that predicts binding affinity corresponding to the protein-ligand binding pose used in the binding energy calculation performed by the first node.

As described above, the first node and the second node may each include a run button. The user may click the run button displayed on the first node to perform the task of calculating the predicted binding energy based on docking between the target protein and ligands. Similarly, the user may click the run button displayed on the second node to perform the task of predicting binding affinity corresponding to the protein-ligand binding pose used in the binding energy calculation performed by the first node.

When the run button displayed on the first node is clicked, the first screen 30 may be displayed. The user may configure the necessary settings to perform the task through the first screen 30. The first screen 30 may include multiple user interface elements. In this specification, the term “user interface elements” refers to all visual components with which the user interacts, such as buttons, labels, text boxes, images, sliders, and drop-down menus. In contrast, the term “user interface controls” specifically refers to components that accept user input and transmit commands to the application, such as buttons, checkboxes, radio buttons, and switches.

The first screen 30 may include multiple user interface elements, including a first user interface element 301. The first user interface element 301 may be used to set the number of results to be displayed after the execution of the task assigned to the first node is completed. For example, if the user enters the value “500” into the first user interface element 301, the predicted binding energies based on docking between the target protein and ligands may be displayed for the top 500 results upon completion of the first task.

Among the multiple user interface elements of the first screen 30, a second user interface element 302 may be used to configure the ligands to be docked with the target protein. For example, the second user interface element 302 may include “Random” and “Upload” as selectable values. If the user selects “Random,” the first task may perform docking between the target protein and ligands based on a ligand library provided by the protein structure-based candidate discovery system itself. Alternatively, if the user selects “Upload,” the user may upload ligand data directly, and the first task may perform docking between the target protein and ligands based on the ligand data uploaded by the user.

Among the multiple user interface elements of the first screen 30, a third user interface element 303 may be used to configure the type and number of ligands to be docked with the target protein. For example, if the user enters “FDA,” “All,” and “2115” into the third user interface element 303, docking may be performed on all 2115 ligands in the FDA-approved drug library. Alternatively, as another example, if the user enters “MCULE,” “In Stock,” and “2000” into the third user interface element 303, docking may be performed on 2000 in-stock compounds from the Mcule library, which are available for immediate shipment.

Among the multiple user interface elements of the first screen 30, a fourth user interface element 304 may be used to configure the number of GPUs to be utilized for the task performed by the first node, which calculates the predicted binding energy based on docking between the target protein and ligands. For example, if the user enters “1” into the fourth user interface element 304, one GPU thread may be used to perform the task. Alternatively, if the user enters “2,” two GPU threads may be used to perform the task.

Among the multiple user interface elements of the first screen 30, a fifth user interface element 305 may display the number of tokens required to execute the task performed by the first node, which calculates the predicted binding energy based on docking between the target protein and ligands. The protein structure-based candidate discovery system is implemented to deduct tokens for detailed simulation tasks related to candidate discovery or to allow users to recharge tokens by paying costs through coupons or various payment methods. The number of tokens deducted is determined based on various factors of the detailed task, such as the type of task, the workload of the task, and the complexity of the task. Data regarding the criteria for token deduction may be stored in a format readable by a computing device in a storage medium or on the cloud, accessible by the computing device. Users may use tokens for the desired detailed tasks based on the established deduction criteria. As previously described, a simulation workflow tailored to optimal compound development may be created by configuring the connections between nodes, each having specific functionalities for candidate discovery. Users may access each node by paying tokens, which are applied differentially for each node, and only pay the required number of tokens for the operations performed by the selected node. For example, the fifth user interface element 305 may indicate that 2,115 tokens are required for the simulation task performed by the first node when using the FDA-approved drug library, corresponding to the number of ligands. If the value set in the fourth user interface element 304 is changed from “1” to “2,” two GPU threads may be allocated to perform the simulation task for 2,115 ligands. This would double the token cost but reduce the execution time by half.

Among the multiple user interface elements of the first screen 30, a sixth user interface element 306 may be used to submit the settings configured through the first screen 30. This submission allows the sequential execution of molecular docking for the plurality of ligands based on the settings entered by the user via the first screen 30, enabling the calculation of the predicted binding energy.

Here, the first user interface element 301, the second user interface element 302, the third user interface element 303, the fourth user interface element 304, and the sixth user interface element 306 may be treated as user interface controls, unlike the fifth user interface element 305.

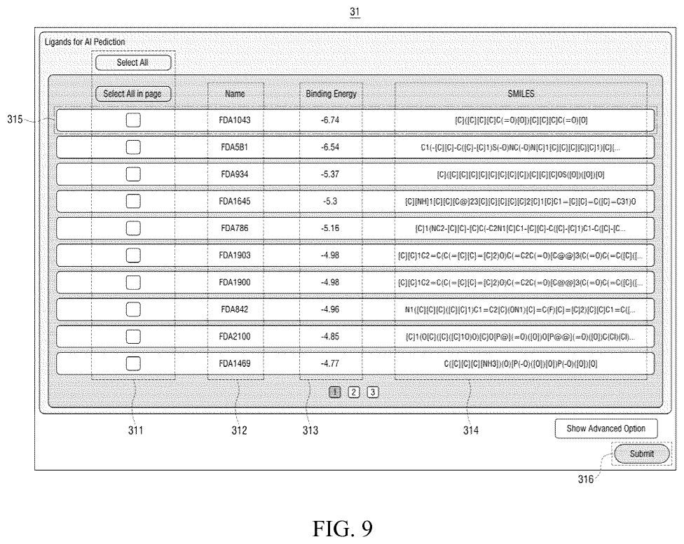

Referring to FIG. 9, a protein structure-based candidate discovery system according to an embodiment may display a second screen 31 that provides the user with the predicted binding energies as results based on docking between the target protein and ligands. As previously described, after the run button displayed on the first node is changed to a result button, the user may click the result button to view the results sorted by binding energy order through the second screen 31. In some embodiments, a download button may additionally be generated on the first node, allowing the user to click the download button to download the results sorted by binding energy order.

The second screen 31 may include multiple user interface elements. Among these, a first user interface element 311 on the second screen 31 may be a user interface control for user selection, such as a checkbox. In some embodiments, for the convenience of selecting multiple rows, a button labeled “Select All in Page” may be additionally displayed to allow the selection of all rows on the current page, or a button labeled “Select All” may be displayed to enable the selection of all results at once.

Among the multiple user interface elements of the second screen 31, a second user interface element 312 may display the names of the plurality of ligands subjected to molecular docking.

Among the multiple user interface elements of the second screen 31, a third user interface element 313 may display the predicted binding energies resulting from the docking of the target protein with the plurality of ligands. Here, the third user interface element 313 may present the binding energy values in units representing the amount of energy. For example, the third user interface element 313 may display the binding energy values in units of Kcal/mol.

Among the multiple user interface elements of the second screen 31, a fourth user interface element 314 may display the structure of the ligands subjected to molecular docking in a string format. For example, the fourth user interface element 314 may represent the structure of the ligands in a string format based on SMILES (Simplified Molecular Input Line Entry System) notation. In SMILES notation, atoms are represented by element symbols, and bonds are represented by specific characters such as ‘=’, #, and others. Additionally, branches within a molecule may be represented using parentheses, ring structures may be denoted by numbers, and chirality may be expressed using specific symbols such as ‘@’.

The second screen 31 displays the results as a first list composed of multiple rows. A single row 315 may include a user interface control for user selection, the names of the plurality of ligands, and the calculated binding energy values.

Among the multiple user interface elements of the second screen 31, a fifth user interface element 316 may be used to submit the information for the rows selected through the first user interface element 311 in the first list of the second screen 31, enabling the calculation of binding affinity corresponding to the protein-ligand binding poses used in the binding energy calculation.

Here, the first user interface element 311 and the fifth user interface element 316 may be treated as user interface controls, unlike the second user interface element 312, the third user interface element 313, and the fourth user interface element 314.

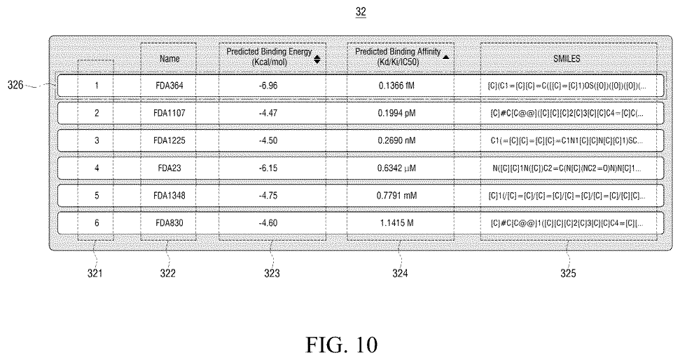

Referring to FIG. 10, a protein structure-based candidate discovery system according to an embodiment may display a third screen 32 that provides the user with predicted binding affinities as results corresponding to the binding energies of the rows selected by the user. The user may click the fifth user interface element 316 on the second screen 32 to view the results sorted by binding affinity order through the third screen 32. Alternatively, if the user navigates from the second screen 32 to the screen displaying the nodes, they may click the result button displayed on the second node to view the results sorted by binding affinity order through the third screen 32. In some embodiments, a download button may additionally be generated on the second node, allowing the user to click the download button to download the results sorted by binding affinity order.

The third screen 32 may include multiple user interface elements. Among these, a first user interface element 321 on the third screen 32 may display the ranking order sorted by binding affinity.

Among the multiple user interface elements of the third screen 32, a second user interface element 322 may display the names of the ligands subjected to molecular docking. Specifically, it may display only those ligands that were shown in the second user interface element 312 of the second screen 31 and selected by the user through the first user interface element 311.

Among the multiple user interface elements of the third screen 32, a third user interface element 323 may display the predicted binding energies resulting from the docking of the target protein with the plurality of ligands. Specifically, it may display only the binding energies that were shown in the third user interface element 313 of the second screen 31 and selected by the user through the first user interface element 311.

Among the multiple user interface elements of the third screen 32, a fourth user interface element 324 may display the binding affinities corresponding to the protein-ligand binding poses used in the binding energy calculation. Here, the fourth user interface element 324 may present the binding affinity values in units of molar concentration. For example, the fourth user interface element 324 may display the binding affinity values in units such as fM, pM, nM, μM, mM, or M.

Among the multiple user interface elements of the third screen 32, a fifth user interface element 315 may display the structures of the ligands subjected to molecular docking in a string format. Specifically, it may display only those ligand structures that were shown in the fourth user interface element 314 of the second screen 31 and selected by the user through the first user interface element 311.

The third screen 32 displays the results as a second list composed of multiple rows. A single row 326 may include the names of the ligands, the binding energy values, and the binding affinity values.

In this embodiment, the rows comprising the second list may be displayed on the third screen sorted in descending or ascending order based on the binding affinity values. Of course, on the same third screen, it may also be possible to sort the rows in descending or ascending order based on the binding energy values.

In this way, by predicting the binding affinity between the target protein and ligands and presenting the binding energy and binding affinity side by side in parallel format—i.e., as adjacent columns on a single screen—the system helps users intuitively understand the results. This allows users to calculate the molecular weight and media volume of candidate compounds, such as drug candidates, without requiring additional experiments, enabling direct application to cell experiments or clinical trials. As a result, the time and cost required to determine the experimental concentration for candidate compounds can be significantly reduced.

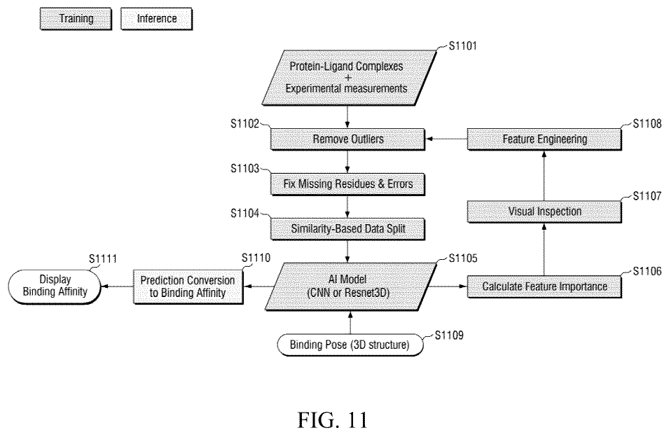

FIG. 11 is a diagram for explaining an operation method of a protein structure-based candidate discovery system according to an embodiment.

A method for operating a protein structure-based candidate discovery system according to an embodiment, implemented as a cloud platform to provide functionalities or services required for protein structure-based candidate discovery in the form of a web service, may include: displaying a first screen for receiving input of a plurality of ligands to be docked to a target protein; calculating predicted binding energy by sequentially performing molecular docking on the plurality of ligands based on settings input by a user via the first screen; displaying a first list on a second screen, the first list comprising rows including a user interface control for user selection, names of the plurality of ligands, and the calculated binding energy values; predicting binding affinity corresponding to protein-ligand binding poses used in the binding energy calculation for the rows selected by the user via the user interface control from the first list displayed on the second screen; and displaying a second list on a third screen, the second list comprising rows including the names of the plurality of ligands, values of the binding energy, and values of the predicted binding affinity. For more details on the method for operating the protein structure-based candidate discovery system, reference can be made to the descriptions provided earlier with reference to FIG. 1 to FIG. 10. Therefore, redundant explanations will be omitted here.

In some embodiments, the binding affinity may be predicted using an artificial intelligence model trained on experimental measurement values, including at least one of the dissociation constant (Kd), inhibition constant (Ki), and half maximal inhibitory concentration (IC50), as training data. Here, the artificial intelligence model may be trained using both the experimental measurement values and the binding structure data of the target protein and ligands as training data. In some embodiments, the artificial intelligence model may include a CNN (Convolutional Neural Network) model or a ResNet 3D (Residual Network 3D) model.

Referring to FIG. 11, the training of the artificial intelligence model may be performed according to steps S1101 through S1108, and the prediction of binding affinity based on the artificial intelligence model may be performed according to steps S1109 through S1111.

In step S1101, the binding structures of proteins and ligands, along with experimentally measured values, may be received as training data. The binding structures of proteins and ligands may be in a three-dimensional format or PDB data, and the experimentally measured values may include at least one of Kd, Ki, and IC50.

In step S1102, outliers present in the training data may be removed. Here, outliers refer to values that are physically unlikely to exist, often due to measurement errors or structural errors in the protein. For example, if outliers are present in the PDBBind dataset, a histogram-based outlier removal technique may be used to eliminate them.

In step S1103, missing residues in the protein structure and ligand may be detected. If missing residues are found, appropriate protein amino acid sequences for completing the missing residues may be obtained through sequence database searches, and the missing residues may be automatically filled using the retrieved protein amino acid sequences. If the protein structure cannot be corrected, it may be removed from the dataset.

In step S1104, datasets may be separated to ensure that similar structures are not mixed into the validation or test datasets. For example, the separation of datasets may be performed based on the TM score (Template Modeling Score), which is an indicator used to evaluate the similarity between predicted three-dimensional protein structures and experimentally determined actual structures.

In step S1105, training of the artificial intelligence model may be performed using the training data prepared through the previous steps.

In steps S1106 through S1108, feature importance may be calculated to explain how each input feature of the artificial intelligence model contributes to the final prediction. This calculation may be based on SHAP (SHapley Additive exPlanations) values, which numerically evaluate the contribution of each input variable to individual predictions. Using protein structure analysis tools, visual inspection and similar methods may be conducted, followed by feature engineering to add or remove features as needed.

In step S1109, the three-dimensional structure pose of the target protein bound with a candidate molecule may be input into the trained artificial intelligence model for prediction. In step S1110, the model may generate a prediction in the form of binding affinity based on molar concentration, making it interpretable for users with only biological knowledge to apply directly in drug formulation. In step S1111, the binding affinity may be displayed to the user on the screen as previously described in relation to FIG. 10.

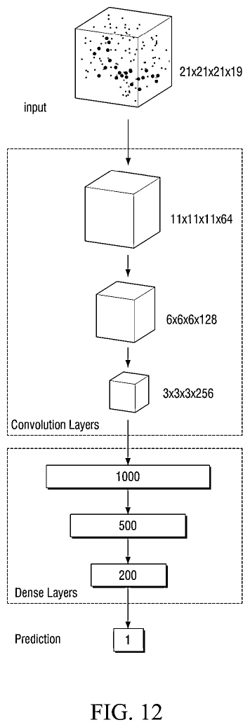

FIG. 12 is a diagram showing an implementation example of the artificial intelligence model of the protein structure-based candidate discovery system according to an embodiment.

Referring to FIG. 12, an artificial intelligence model for predicting binding affinity in a protein structure-based candidate discovery system according to an embodiment may include a convolution layer part and a dense layer part. The convolution layer part may include filters that encode patterns to be identified for predicting the binding affinity between the target protein and the ligand. Meanwhile, the dense layer part may integrate the features extracted by the convolution layer part.

In some embodiments, the artificial intelligence model may be a deep 4D convolutional neural network, specifically a deep 4D convolutional neural network with a single output neuron designed to predict the binding affinity between the target protein and the ligand.

The convolution layer part may identify patterns encoded by the filters of the convolution layers and generate feature maps that emphasize the spatial occurrences of each pattern within the data. Specifically, the convolution layer part may include three convolution layers with 64, 128, and 256 filters, respectively. The output of the final convolution layer may be flattened and used as input for the dense layer part.

The dense layer part may include three dense layers with 1000, 500, and 200 neurons, respectively. A dropout with a drop probability of 0.5 may be applied to all dense layers. Additionally, an L2 regularization technique with a parameter lambda value of 0.001 may be used.

Meanwhile, both the convolution layer part and the dense layer part may adopt ReLU (Rectified Linear Unit) as the activation function.

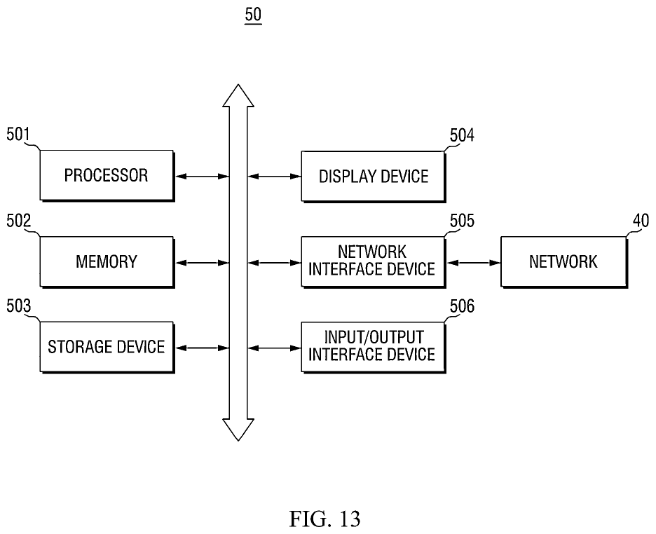

FIG. 13 is a block diagram for explaining a computing device according to an embodiment.

Referring to FIG. 13, a protein structure-based candidate discovery system according to the embodiments may be implemented using a computing device 50.

The computing device 50 may include, via communication through a bus 509, at least one of the following: a processor 501, a memory 502, a storage device 503, a display device 504, a network interface device 505 providing access to a network 40 for communication with other entities, and an input/output interface device 506 providing user input or output interfaces. Of course, the computing device 50 may also include any additional electronic devices necessary to implement the technical ideas described in this specification, even if not shown in FIG. 13.

The processor 501 may be implemented as various types of devices, such as an application processor (AP), central processing unit (CPU), graphics processing unit (GPU), or neural processing unit (NPU). It may be any electronic device capable of executing programs or instructions stored in the memory 502 or the storage device 503. Specifically, the processor 501 may be configured to implement the functions or methods described earlier in relation to FIG. 1 through FIG. 12. For the protein structure-based candidate discovery system and its operation method according to the embodiments of the present invention, Al-specialized computations may be processed on the GPU or NPU.

The memory 502 and the storage device 503 may include various types of volatile or non-volatile storage media. For example, the memory 502 may include ROM (read-only memory) or RAM (random access memory). The memory 502 may be located internally or externally to the processor 501 and may be connected to the processor 501 through various known means. Examples of the storage device 503 include HDDs (Hard Disk Drives) or SSDs (Solid State Drives), among others. The scope of the present invention is not limited to the elements listed above for illustrative purposes.

The protein structure-based candidate discovery system and its operation method according to the embodiments may be implemented as programs or software executed on the computing device 50. Such programs or software may be stored on a computer-readable medium.

Meanwhile, the protein structure-based candidate discovery system and its operation method according to the embodiments may be implemented using the hardware of the computing device 50 or as separate hardware that can be electrically connected to the computing device 50.

According to the embodiments described thus far, the system provides functionalities and user interfaces optimized for candidate discovery. It addresses the challenges of conventional methods, where detailed tasks related to candidate discovery were provided as separate tools with low compatibility, making data sharing and consistent data management difficult. Furthermore, it allows easy management of simulation workflows through the creation, modification, and deletion of nodes.

In conjunction with this, the system predicts and provides the binding affinity between the target protein and ligands to the user, facilitating an intuitive understanding of the results. This enables users to easily calculate essential parameters, such as the molecular weight of candidate compounds (e.g., drug candidates) and the media volume, without requiring additional experiments, allowing direct application to cell experiments or clinical trials. As a result, the time and cost associated with determining experimental concentrations for candidate compounds can be significantly reduced. Moreover, since these experimental concentrations are provided through a cloud platform, users can conveniently access the concentration values from anywhere with an internet connection, and collaboration with other users becomes more seamless.

The embodiments of the present invention have been described in detail above, but the scope of the present invention is not limited to these descriptions. Various modifications and improvements that utilize the basic concepts of the present invention, as defined in the following claims, and that are made by those skilled in the art to which the present invention pertains, are also within the scope of the present invention.

1. A method for operating a protein structure-based candidate discovery system implemented as a cloud platform to provide functionalities or services required for protein structure-based candidate discovery in a form of a web service, the method comprising:
displaying a simulation setup area to a user via a display device;
transforming and placing a first object from a task module selection area of the simulation setup area into a first node in a canvas area of the simulation setup area by being dragged and dropped, wherein the first node calculates binding energy predicted from docking between a target protein and a ligand;
transforming and placing a second object from the task module selection area of the simulation setup area into a second node in the canvas area of the simulation setup area by being dragged and dropped, wherein the second node predicts binding affinity corresponding to protein-ligand binding pose used in the binding energy calculation performed by the first node;
connecting the first node and the second node with an edge such that the second node follows the first node;
displaying a first screen for receiving input of a plurality of ligands to be docked to the target protein, when a run button displayed on the first node is clicked;
calculating predicted binding energy by sequentially performing molecular docking on the plurality of ligands based on settings input by the user via the first screen, when the settings configured through the first screen are submitted;
displaying a first list on a second screen, the first list comprising rows including a user interface control for user selection, names of the plurality of ligands, and the calculated binding energy values, when the run button on the first node is changed to a result button and the result button is clicked;
predicting binding affinity corresponding to protein-ligand binding poses used in the binding energy calculation for the rows selected by the user via the user interface control from the first list displayed on the second screen, when the settings configured through the second screen are submitted; and
displaying a second list on a third screen, the second list comprising rows including the names of the plurality of ligands, values of the binding energy, and values of the predicted binding affinity, when the result button displayed on the second node is clicked.

2. The method of claim 1, wherein
the values of the binding energy are displayed in a unit representing the amount of energy, and
the values of the binding affinity are displayed in a unit of molar concentration.

3. The method of claim 2, wherein
the values of the binding energy are displayed in a unit of Kcal/mol, and
the values of the binding affinity are displayed in a unit of fM, pM, nM, μM, mM, or M.

4. The method of claim 3, wherein the displaying the second list on the third screen comprises:
displaying the rows constituting the second list on the third screen, sorted in descending or ascending order based on the values of the binding affinity.

5. The method of claim 2, wherein the binding affinity is predicted using an artificial intelligence model, the artificial intelligence model being trained with experimental measurement data including at least one of a dissociation constant (Kd), an inhibition constant (Ki), and a half maximal inhibitory concentration (IC50).

6. The method of claim 5, wherein the artificial intelligence model is trained using the experimental measurement data and binding structure data of the target protein and the ligand as training data.

7. The method of claim 6, wherein the artificial intelligence model comprises a convolution layer part including filters for encoding patterns to be identified for predicting the binding affinity between the target protein and the ligand, and a dense layer part for integrating features extracted through the convolution layer part.

8. The method of claim 7, wherein the convolution layer part comprises three convolution layers, each having 64, 128, and 256 filters, respectively.

9. The method of claim 8, wherein the dense layer part comprises three dense layers, each having 1000, 500, and 200 neurons, respectively.

10. The method of claim 5, wherein the artificial intelligence model comprises a CNN(Convolutional Neural Network) model or a ResNet 3D (Residual Network 3D) model.

11. A protein structure-based candidate discovery system implemented as a cloud platform to provide functionalities or services required for protein structure-based candidate discovery in a form of a web service, the system comprising:
a project management module configured to create a project for adding a task to perform the discovery of a protein structure-based candidate;
a simulation management module configured to create a simulation desired by a user on the created project;
a simulation setting module configured to set a simulation workflow for the simulation based on user input using a simulation setting area, the simulation setting area comprising a task module selection area, the task module selection area comprising:
a first object, which is dragged and dropped onto a canvas area and converted into a first node that calculates predicted binding energy based on docking between a target protein and a ligand, and
a second object, which is dragged and dropped onto the canvas area and converted into a second node that predicts binding affinity corresponding to protein-ligand binding pose used in the binding energy calculation performed by the first node; and
a simulation workflow management module configured to manage information on nodes that can precede or follow in the simulation workflow,
wherein:
an edge is connected between the first node and the second node such that the second node follows the first node,
when a run button displayed on the first node is clicked, a first screen is displayed to receive a plurality of ligands to be docked with the target protein,
when the settings configured through the first screen are submitted, predicted binding energy is calculated by sequentially performing molecular docking on the plurality of ligands based on the settings input by the user through the first screen,
when the run button displayed on the first node is changed to a result button and the result button is clicked, a first list comprising rows including a user interface control for user selection, names of the plurality of ligands, and the calculated binding energy values is displayed on a second screen,
when the settings configured through the second screen are submitted, binding affinity corresponding to protein-ligand binding pose used in the binding energy calculation is predicted for the rows selected by the user via the user interface control from the first list of the second screen, and
when the result button displayed on the second node is clicked, a second list comprising rows including the names of the plurality of ligands, values of the binding energy, and values of the predicted binding affinity is displayed on a third screen.

12-16. (canceled)

17. The system of claim 1, wherein the second list is displayed on the third screen by displaying the rows constituting the second list on the third screen, sorted in descending or ascending order based on the values of the binding affinity.

18. The system of claim 11, wherein
the values of the binding energy are displayed in a unit of Kcal/mol, and
the values of the binding affinity are displayed in a unit of fM, pM, nM, μM, mM, or M.

19. The system of claim 11, wherein the binding affinity is predicted using an artificial intelligence model, the artificial intelligence model being trained with experimental measurement data including at least one of a dissociation constant (Kd), an inhibition constant (Ki), and a half maximal inhibitory concentration (IC50).

20. The system of claim 11, wherein the simulation workflow management module manages information on nodes that can precede or follow in the simulation workflow through metadata.

21. The system of claim 19, wherein the artificial intelligence model is trained using the experimental measurement data and binding structure data of the target protein and the ligand as training data.

22. The system of claim 21, wherein the artificial intelligence model comprises a convolution layer part including filters for encoding patterns to be identified for predicting the binding affinity between the target protein and the ligand, and a dense layer part for integrating features extracted through the convolution layer part.

23. The system of claim 22, wherein the convolution layer part comprises three convolution layers, each having 64, 128, and 256 filters, respectively.

24. The system of claim 23, wherein the dense layer part comprises three dense layers, each having 1000, 500, and 200 neurons, respectively.

25. The system of claim 19, wherein the artificial intelligence model comprises a CNN(Convolutional Neural Network) model or a ResNet 3D (Residual Network 3D) model.
